(19) United States
(12) Patent Application Publication
(10) Pub. No.: US 2025/0250736 A1
(43) Pub. Date: Aug. 7, 2025

(54) APPAREL VAPORIZING DEVICE CONFIGURED FOR VAPORIZING APPAREL WITH CUSTOMIZED NATURAL AROMAS OR NUTRIENTS WHICH CREATES THE PROCESS OF THERAPEUTIC VAPORIZED APPAREL, AND A METHOD OF USE THEREOF

(71) Applicant: Dustin Jenkins, Charlotte, NC (US)

(72) Inventor: Dustin Jenkins, Charlotte, NC (US)

(21) Appl. No.: 18/434,239

(22) Filed: Feb. 6, 2024

(51) Int. Cl. D06M 13/00; A61K 9/00; A61K 41/00; D06B 5/24; D06B 23/04; D06B 23/14; D06M 23/06

(52) U.S. Cl. CPC D06M 13/005; A61K 9/0078; A61K 41/0004; D06B 5/24; D06B 23/04; D06B 23/14; D06M 23/06; D10B 2501/00; D10B 2509/00

(57) ABSTRACT

An apparel vaporizing device is for creating vaporized apparel. The apparel vaporizing device includes an enclosure with an inside configured to hold at least one apparel item. A hanging mechanism is positioned inside of the enclosure. The hanging mechanism is configured for hanging at least one of the at least one apparel item thereon in the inside of the enclosure. A vapor container is in communication with the inside of the enclosure. The vapor container is configured for providing a vapor. A pump is configured to move the vapor from the vapor container throughout the inside of the enclosure and onto each of the at least one apparel item inside of the enclosure.

### FIELD OF THE DISCLOSURE

The present disclosure is directed to a homeopathic method for the creation of aromas and nutrients made applicable to various articles of apparel with the intent to influence a therapeutic effect upon the occupant.

### BACKGROUND

Generally speaking, aromatherapy is based on the use of aromatic materials, including essential oils and other aroma compounds, for improving psychological well-being. Aromatherapy is known to be offered as a complementary therapy or as a form of alternative medicine. People use blends of essential oils as topical application, massage, inhalation, or water immersion. There is evidence that aromatherapy may be effective in combating postoperative nausea and vomiting.

A diffuser, or aroma lamp, may be used to diffuse essential oils for aromatherapy purposes. The diffuser may project the oil into the air by heating it, letting it evaporate naturally, or nebulizing it using compressed air or ultrasonics. An evaporative diffuser is a device that uses a pad, filter, or reeds to diffuse the essential oils. One of the disadvantages of this tool is that the light elements of the essential oils will be circulated around the ceiling first and will only come down at the end of the process. A heat diffuser contains a small candle under a bowl to vaporize a mixture of water and oil. This device is very cheap and doesn't need a special maintenance, however, the heat can change the chemical structure of the oils. Certain plug-in nightlight units made by companies such as Air Wick are used in combination with glass bulbs of scented oil, filling a room with fragrance and light. A nebulizer is a device that does not use water or heat as other diffusers. The bottle of essential oil is attached directly to the nebulizer. As a result, the unit uses 100% pure essential oil. The benefit of using this device is a strong concentration of the essential oil. However, it can be noisier than the other devices depending on the specific model in use. An ultrasonic diffuser is the same as a nebulizer but uses a water/oil mixture. This makes the mist not so concentrated. This device can be used as a humidifier. These diffusers contain an ultrasonic transducer, which vaporizes the water/oil mixture through cavitation and creates instantaneous vapor.

Aromatherapy, and such diffuser devices for providing aromatherapy, are generally done at home or in spas or like establishments. Currently, there is no known device and/or method for making aromatherapy portable or wearable so that it is affective beyond the point of use at home, in a spa, or in other like establishments. Therefore, a need exists for providing a mechanism and/or method for making aromatherapy portable or wearable.

The instant disclosure may be designed to address at least certain aspects of the problems or needs discussed above by providing an apparel vaporizing device configured for vaporizing apparel with customized natural aromas or nutrients which creates the process of therapeutic vaporized apparel, and a method of use thereof.

### SUMMARY

The present disclosure may solve the aforementioned limitations of the currently available aromatherapy devices or methods of providing aromatherapy by providing an apparel vaporizing device. The disclosed apparel vaporizing device may be for creating vaporized apparel. The apparel vaporizing device may include an enclosure with an inside configured to hold at least one apparel item. A hanging mechanism may be positioned inside of the enclosure. The hanging mechanism may be configured for hanging at least one of the at least one apparel items thereon in the inside of the enclosure. A vapor container may be in communication with the inside of the enclosure. The vapor container may be configured for providing a vapor. A pump may be configured to move the vapor from the vapor container throughout the inside of the enclosure and onto each of the at least one apparel items inside of the enclosure.

One feature of the disclosed apparel vaporizing device may be that it can be configured to vaporize each of the at least one apparel items inside of the enclosure via the vapor from the vapor container.

Another feature of the disclosed apparel vaporizing device may be that the vapor from the vapor container may be configured to vaporize each of the at least one apparel items with customized natural aromas, nutrients, or a combination thereof.

Another feature of the disclosed apparel vaporizing device may be that the vapor from the vapor container may be configured to create therapeutic vaporized apparel from each of the at least one apparel items inside the enclosure.

Another feature of the disclosed apparel vaporizing device may be that the vapor from the vapor container may be configured to provide a deodorizer for each of the at least one apparel items inside of the enclosure.

Another feature of the disclosed apparel vaporizing device may be that the vapor from the vapor container may be configured to provide an alternative dry-cleaning of each of the at least one apparel items inside of the enclosure.

Another feature of the disclosed apparel vaporizing device may be that the vapor from the vapor container may be configured to provide organic infusion to each of the at least one apparel items inside of the enclosure.

In select embodiments of the disclosed apparel vaporizing device, the enclosure may be a wooden cabinet structure.

In select embodiments of the disclosed apparel vaporizing device, the enclosure may include a width, a height, and a depth. Wherein, the width, the height, and the depth may be sized to accommodate each of the at least one apparel items hung on the hanging mechanism in the enclosure. In select embodiments, the enclosure may include the width of approximately 4 feet, the height of approximately 6 feet, or the depth of approximately 2.5 feet. In select possibly preferred embodiments, the enclosure may include the width of 4 feet, the height of 6 feet, and the depth of 2.5 feet.

Another feature of the disclosed apparel vaporizing device may be that the enclosure may include at least one door. The at least one door may be configured to open for accessing the inside of the enclosure, like for inserting or removing each of the at least one apparel items. The at least one door may also be configured to close for sealing the inside of the enclosure for vaporizing each of the at least one apparel items on the inside of the enclosure. In select embodiments of the disclosed apparel vaporizing device, the at least one door may include two (2) outswing style doors on a front side of the enclosure. In other select embodiments of the disclosed apparel vaporizing device, each of the at least one doors may be made of a transparent material. The transparent material may be configured for viewing each of the at least one apparel items positioned on the inside of the enclosure. The transparent material of the doors may be any transparent material, including but not limited to, glass. In select embodiments of the disclosed apparel vaporizing device, the at least one door may extend from approximate a top of the enclosure to a mid-portion of the enclosure. Wherein, a bottom portion of the enclosure does not include a door or opening. Wherein, the bottom portion may be configured for holding non-hanging apparel items that rest on a bottom of the enclosure. In select embodiments of the apparel vaporizing device, the front side of the enclosure may include a front member extending from the bottom of the enclosure up to the mid-portion of the enclosure.

Another feature of the disclosed apparel vaporizing device may be that the hanging mechanism may be a rod, like a clothes hanging rod or closet rod. The rod may be positioned approximate a top of the enclosure and may extend from a left side of the enclosure to a right side of the enclosure.

In select embodiments of the disclosed apparel vaporizing device, the vapor container may be positioned on an outside of the enclosure. In these embodiments, the vapor container may communicate with the inside of the enclosure through a vapor port. In select embodiments, the pump may be positioned in the vapor port for moving the vapor from the vapor container through the vapor port and into the inside of the enclosure. The pump may be any device or mechanism for moving vapor from the vapor container through the vapor port and into the inside of the enclosure. In select embodiments, the pump may be a propeller fan positioned in the vapor port.

In select embodiments of the disclosed apparel vaporizing device, the vapor container may be positioned on an exterior shelf mounted on the outside of the enclosure. The exterior shelf may include a housing configured to seal the vapor container inside of the housing on the exterior shelf mounted on the outside of the enclosure. In select embodiments, the housing of the exterior shelf may include an access panel on one side of the housing. The access panel may be configured to open for accessing the inside of the housing for inserting, removing, or re-filing the vapor container. The access panel may also be configured to close for sealing the inside of the housing with the vapor container therein. In select embodiments, the access panel may be made from a transparent material. The transparent material of the access panel may be any transparent material, including, but not limited to, glass. In select embodiments of the disclosed apparel vaporizing device, the exterior shelf may include an angled support bracket. The angled support bracket may be configured to support the exterior shelf on the outside of the enclosure.

Another feature of the disclosed apparel vaporizing device may be that the vapor container can be any device for providing vapor, including, but not limited to, an aroma lamp, an evaporative diffuser, a heat diffuser, a nebulizer, or an ultrasonic diffuser. In select possibly preferred embodiments, the vapor container may be an ultrasonic diffuser.

Another feature of the disclosed apparel vaporizing device may be that it can further include a low voltage battery. The low voltage battery may be configured to power the vapor container and the pump for moving the vapor from the vapor container throughout the inside of the enclosure and onto each of the at least one apparel items inside of the enclosure.

In another aspect, the instant disclosure embraces the disclosed apparel vaporizing device in any embodiment and/or combination of embodiments shown and/or described herein.

In another aspect, the instant disclosure embraces a method of vaporizing apparel. The disclosed method of vaporizing apparel may generally include providing the disclosed apparel vaporizing device in any embodiment and/or combination of embodiments shown and/or described herein. With the provided apparel vaporizing device, the method of vaporizing apparel may further include inserting the at least one apparel item into the inside of the enclosure; and vaporizing the at least one apparel item by moving vapor from the vapor container throughout the inside of the enclosure.

In select embodiments of the disclosed method of vaporizing apparel, the step of inserting of the at least one apparel item into the inside of the enclosure may include hanging at least one of the at least one apparel items on the hanging mechanism inside of the enclosure.

One feature of the disclosed method of vaporizing apparel may be that it can be used for creating customized natural aromas or nutrients which create the process of therapeutic vaporized apparel. As such, with the disclosed method of vaporizing apparel, a homeopathic method may be provided for the creation of aromas and nutrients made applicable to various articles of apparel with the intent to influence a therapeutic effect upon the occupant.

Another feature of the disclosed method of vaporizing apparel may be that it can be used for allowing the occupant to have the ability to interact with their sense of smell and taste, giving the occupant a tangible mechanism to stimulate behavior and taste, wherein the occupant has the ability to internalize nutrients through absorption and inhalation, and wherein, the occupant has the ability to externalize nutrients which is utilized for destabilizing airborne pathogens, smoke and bacteria hands-free.

The foregoing illustrative summary, as well as other exemplary objectives and/or advantages of the disclosure, and the manner in which the same are accomplished, are further explained within the following detailed description and its accompanying drawings.

### BRIEF DESCRIPTION OF THE DRAWINGS

The present apparatuses, systems and methods will be better understood by reading the Detailed Description with reference to the accompanying drawings, which are not necessarily drawn to scale, and in which like reference numerals denote similar structure and refer to like elements throughout, and in which:

FIG. 1 is a front left perspective view of the disclosed apparel vaporizing device according to select embodiments of the instant disclosure, showing the wooden enclosure with 1 glass door open showing the cylinder rod configured for hanging the apparel and the modified diffuser and modified propeller on the left side of the wooden enclosure;

FIG. 2 is a front bottom right perspective partially broken away view of the disclosed apparel vaporizing device from FIG. 1;

FIG. 3 is a front top right perspective view of the disclosed apparel vaporizing device from FIG. 1 with both glass doors shut;

FIG. 4 is a left side view of the disclosed apparel vaporizing device from FIG. 1 showing the modified diffuser and modified propeller on the left side of the wooden enclosure;

FIG. 5 is a zoomed in left top perspective view of the disclosed apparel vaporizing device from FIG. 1 showing a zoomed in view of the vapor container and pump on the left side of the wooden enclosure showing how vapor moves from the vapor container through the vapor port and into the inside of the enclosure; and

FIG. 6 is a flow chart of the disclosed method of vaporizing apparel according to select embodiments of the instant disclosure.

It is to be noted that the drawings presented are intended solely for the purpose of illustration and that they are, therefore, neither desired nor intended to limit the disclosure to any or all of the exact details of construction shown, except insofar as they may be deemed essential to the claimed disclosure.

### DETAILED DESCRIPTION

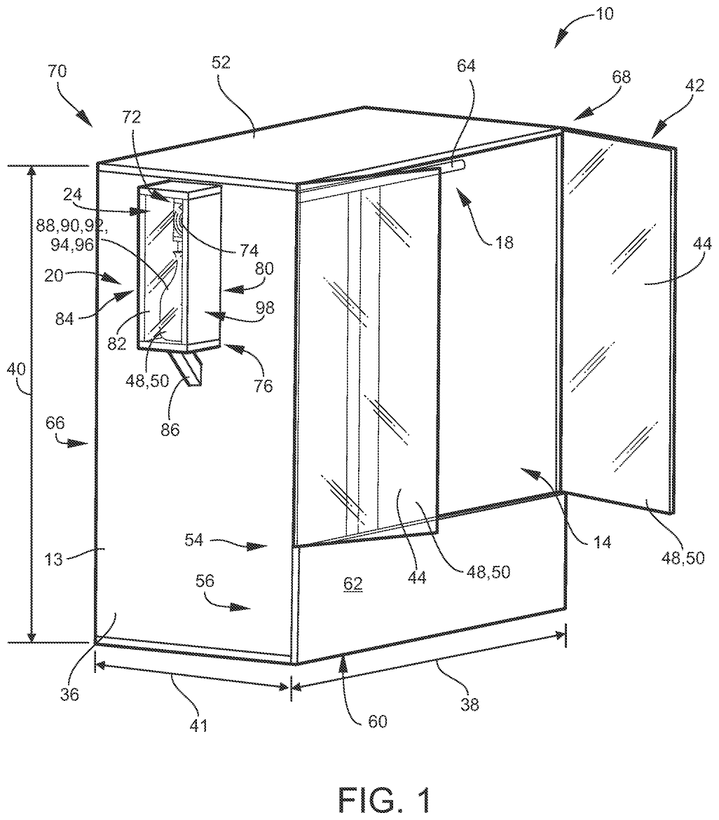

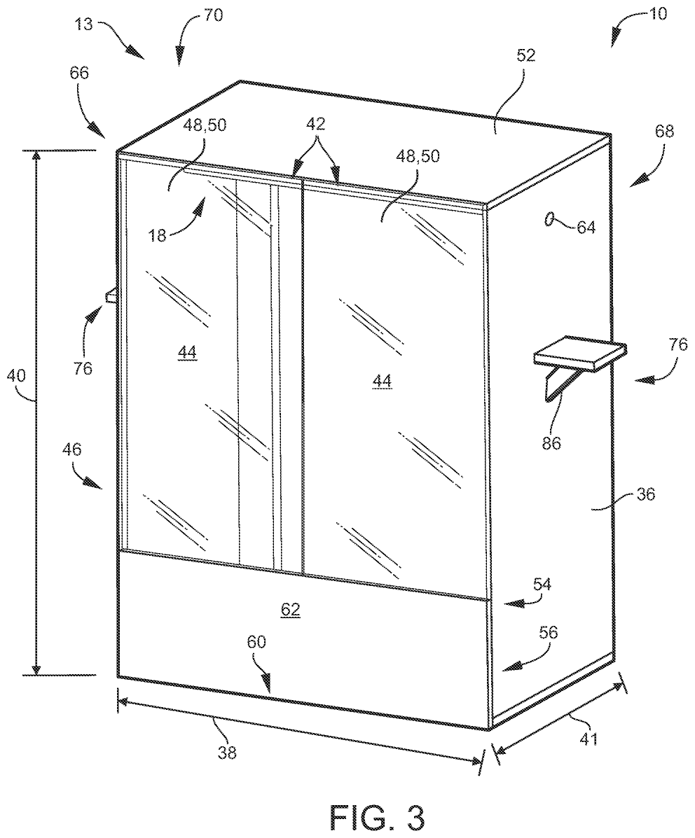

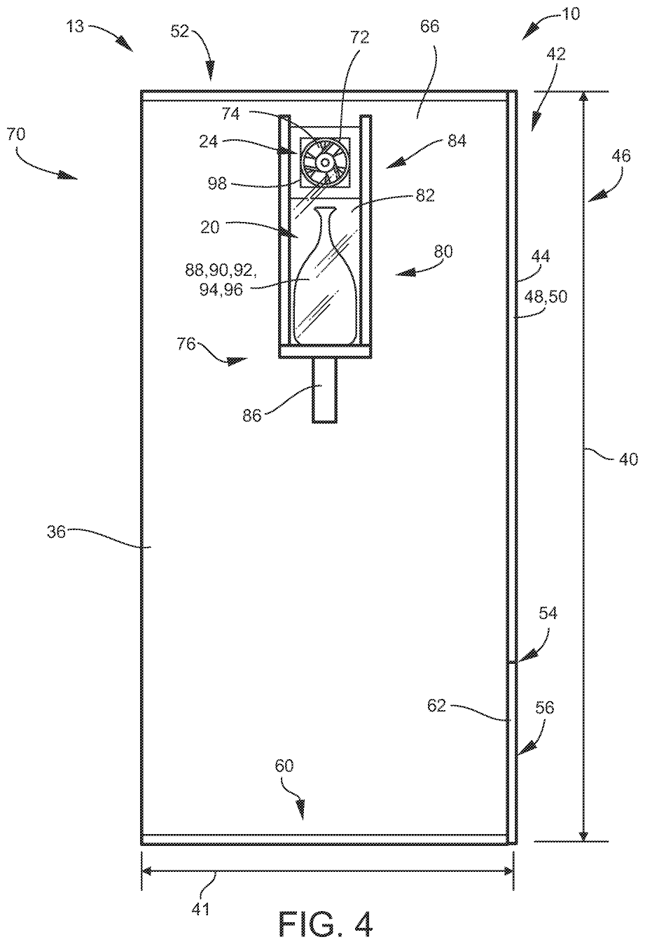

Referring now to FIGS. 1-6, in describing the exemplary embodiments of the present disclosure, specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples and are merely examples among other possible examples.

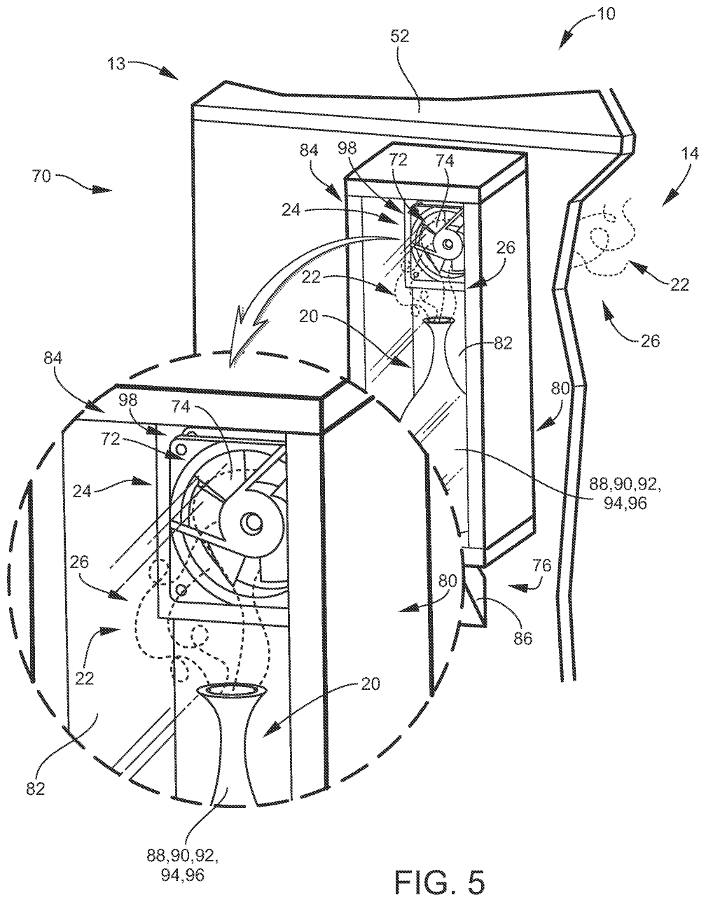

Referring now to FIGS. 1-5, the present disclosure may solve the aforementioned limitations of the currently available aromatherapy devices or methods of providing aromatherapy by providing apparel vaporizing device 10. Apparel vaporizing device 10 may be for creating vaporized apparel. Apparel vaporizing device 10 may include enclosure 13 with inside 14 configured to hold at least one apparel item. Hanging mechanism 18 may be positioned inside of enclosure 13. Hanging mechanism 18 may be configured for hanging at least one of the at least one apparel items thereon in inside 14 of enclosure 13. Vapor container 20 may be in communication with inside 14 of enclosure 13. Vapor container 20 may be configured for providing vapor 22 (see FIG. 5). Pump 24 may be configured to move vapor 22 from vapor container 20 throughout inside 14 of enclosure 13 and onto each of the at least one apparel items inside of enclosure 13.

As such, one feature of apparel vaporizing device 10 may be that it can be configured to vaporize each of the at least one apparel items in inside 14 of enclosure 13 via vapor 22 from vapor container 20. Another feature of apparel vaporizing device 10 may be that vapor 22 from vapor container 20 may be configured to vaporize each of the at least one apparel items with customized natural aromas, nutrients, or a combination thereof. Another feature of apparel vaporizing device 10 may be that vapor 22 from vapor container 20 may be configured to create therapeutic vaporized apparel from each of the at least one apparel items in inside 14 of enclosure 13. Another feature of apparel vaporizing device 10 may be that vapor 22 from vapor container 20 may be configured to provide a deodorizer for each of the at least one apparel items in inside 14 of enclosure 13. Another feature of apparel vaporizing device 10 may be that vapor 22 from vapor container 20 may be configured to provide an alternative dry-cleaning method or means for each of the at least one apparel items in inside 14 of enclosure 13. Another feature of apparel vaporizing device 10 may be that vapor 22 from vapor container 20 may be configured to provide organic infusion to each of the at least one apparel items in inside 14 of enclosure 13.

As shown in FIGS. 1-5, in select embodiments of apparel vaporizing device 10, enclosure 13 may be wooden cabinet structure 36. In select embodiments of apparel vaporizing device 10, enclosure 13 may include width 38, height 40, and depth 41. Wherein, width 38, height 40, and depth 41 may be sized to accommodate each of the at least one apparel items hung on hanging mechanism 18 in inside 14 of enclosure 13, or placed on bottom 60 in inside 14 of enclosure 13, or placed on any other shelves or hangers in inside 14 of enclosure 13. As such, enclosure 13 may be designed and configured with different sizes, shapes and configurations to hold various shapes, sizes and amounts of apparel items in inside 14 of enclosure 13. In select embodiments, enclosure 13 may include width 38 of approximately 4 feet, height 40 of approximately 6 feet, or depth 41 of approximately 2.5 feet. In select possibly preferred embodiments, enclosure 13 may include width 38 of 4 feet, height 40 of 6 feet, and depth 41 of 2.5 feet.

As best shown in FIGS. 1-3, another feature of apparel vaporizing device 10 may be that enclosure 13 may include at least one door 42 The at least one door 42 may be configured to open for accessing inside 14 of enclosure 13, like for inserting or removing each of the at least one apparel items. The at least one door 42 may also be configured to close for sealing inside 14 of enclosure 13 for vaporizing each of the at least one apparel items in inside 14 of enclosure 13. In select embodiments of apparel vaporizing device 10, the at least one door 42 may include two (2) outswing style doors 44 on front side 46 of enclosure 13. In other select embodiments of apparel vaporizing device 10, each of the at least one doors 42 may be made of transparent material 48. Transparent material 48 may be configured for viewing each of the at least one apparel items positioned in inside 14 of enclosure 13. Transparent material 48 of doors 42 may be any transparent material, including but not limited to, glass 50, as shown in the Figures. In select embodiments of apparel vaporizing device 10, the at least one door 42 may extend from approximate top 52 of enclosure 13 to mid-portion 54 of enclosure 13. Wherein, bottom portion 56 of enclosure 13 does not include a door or opening. Wherein, bottom portion 56 may be configured for holding non-hanging apparel items that rest on bottom 60 of enclosure 13. In select embodiments of apparel vaporizing device 10, front side 46 of enclosure 13 may include front member 62 extending from bottom 60 of enclosure 13 up to mid-portion 54 of enclosure 13.

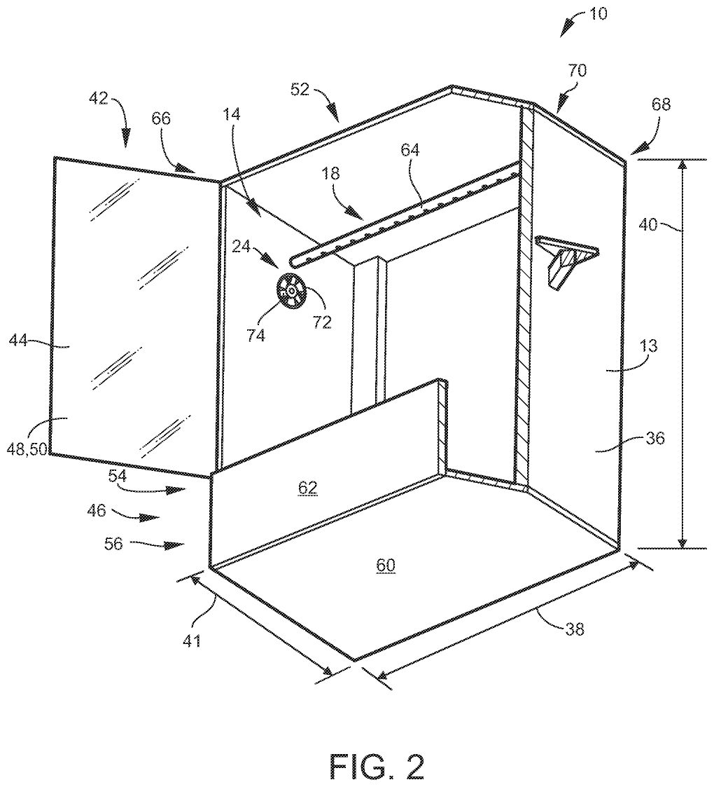

As best shown in FIGS. 1-2, another feature of apparel vaporizing device 10 may be that hanging mechanism 18 may be, but is not limited to, rod 64. Rod 64 may be any rod type device, like a clothes hanging rod or closet rod. Rod 64 may be positioned approximate top 52 of enclosure 13 and may extend from left side 66 of enclosure 13 to right side 68 of enclosure 13.

As best shown in FIGS. 1, 4 and 5, in select embodiments of apparel vaporizing device 10, vapor container 20 may be positioned on outside 70 of enclosure 13. In these embodiments, vapor container 20 may communicate with inside 14 of enclosure 13 through vapor port 72. Vapor port 72 may be a hole through a member of enclosure 13, like a left side member (as shown in the Figures), a right side member, a top member, and/or even a bottom member. In select embodiments, pump 24 may be positioned in vapor port 72 for moving vapor 22 from vapor container 20 through vapor port 72 and into inside 14 of enclosure 13. Pump 24 may be any device or mechanism for moving vapor 22 from vapor container 20 through vapor port 72 and into inside 14 of enclosure 13. In select embodiments, as shown best in FIG. 5, pump 24 may be a propeller fan 74 positioned in vapor port 72 configured for moving vapor 22 from vapor container 20 through vapor port 72 and into inside 14 of enclosure 13.

As best shown in FIG. 1, in select embodiments of apparel vaporizing device 10, vapor container 20 may be positioned on exterior shelf 76. Exterior shelf 76 may be mounted on outside 70 of enclosure 13. Exterior shelf 76 may include housing 80 configured to seal vapor container 20 inside of housing 80 on exterior shelf 76 mounted on outside 70 of enclosure 13. In select embodiments, housing 80 of exterior shelf 76 may include access panel 82 on one side 84 of housing 80. Access panel 82 may be configured to open for accessing the inside of housing 80 for inserting, removing, or re-filing vapor container 20. Access panel 82 may also be configured to close for sealing the inside of housing 80 with vapor container 20 therein. In select embodiments, access panel 82 may be made from transparent material 48. Transparent material 48 of access panel 82 may be any transparent material, including, but not limited to, glass 50. In select embodiments of apparel vaporizing device 10, exterior shelf 76 may include angled support bracket 86. Angled support bracket 86 may be configured to support exterior shelf 76 on outside 70 of enclosure 13.

Another feature of apparel vaporizing device 10 may be that vapor container 20 can be any device for providing vapor 22, including, but not limited to, aroma lamp 88, evaporative diffuser 90, heat diffuser 92, nebulizer 94, ultrasonic diffuser 96, the like, or any various combinations thereof. In select possibly preferred embodiments, vapor container 20 may be ultrasonic diffuser 96.

Another feature of apparel vaporizing device 10 may be that it can further include low voltage battery 98. Low voltage battery 98, or the like, may be configured to power vapor container 20 (like any aroma lamp 88, evaporative diffuser 90, heat diffuser 92, nebulizer 94, ultrasonic diffuser 96, the like, or any various combinations thereof) and pump 24 (like propeller fan 74, or the like) for moving vapor 22 from vapor container 20 throughout inside 14 of enclosure 13 and onto each of the at least one apparel items in inside 14 of enclosure 13. However, the instant disclosure is not so limited to a battery powered apparel vaporizing device 10, and apparel vaporizing device 10 may be powered by any other means, like any form of electrical power.

In another aspect, the instant disclosure embraces the disclosed apparel vaporizing device in any embodiment and/or combination of embodiments shown and/or described herein.

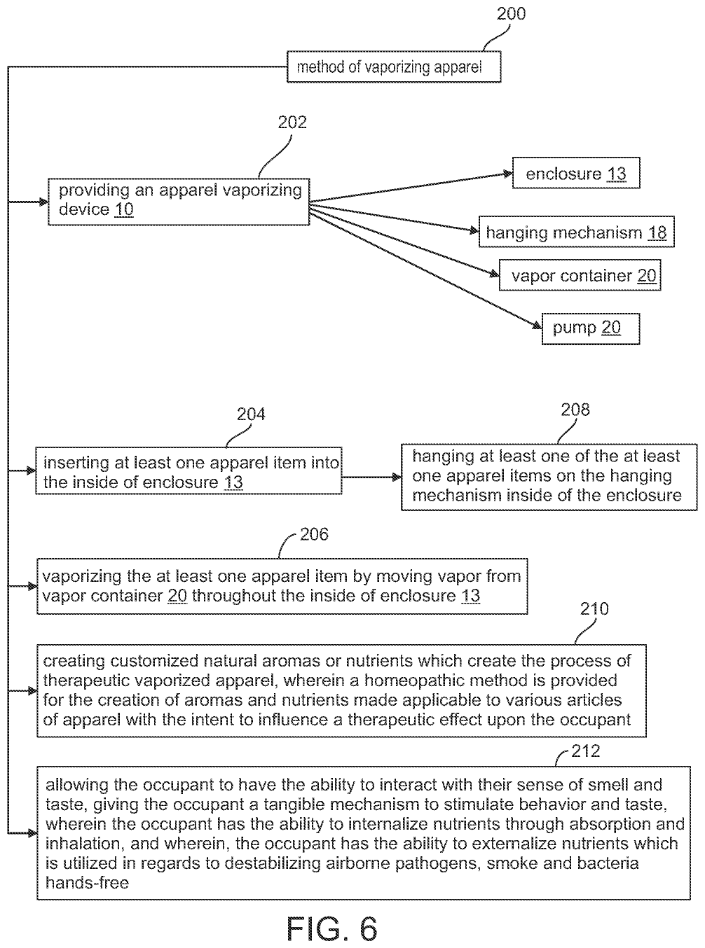

Referring now to FIG. 6, in another aspect, the instant disclosure embraces method 200 of vaporizing apparel. The disclosed method 200 of vaporizing apparel may generally include step 202 of providing the disclosed apparel vaporizing device 10 in any embodiment and/or combination of embodiments shown and/or described herein. With the provided apparel vaporizing device 10, method 200 of vaporizing apparel may further include step 204 of inserting the at least one apparel item into inside 14 of enclosure 13; and step 206 of vaporizing the at least one apparel item by moving vapor 22 from vapor container 20 throughout inside 14 of enclosure 13. In select embodiments of method 200 of vaporizing apparel, step 204 of inserting of the at least one apparel item into inside 14 of enclosure 13 may include step 208 of hanging at least one of the at least one apparel items on hanging mechanism 18 (like rod 64) in inside 14 of enclosure 13.

One feature of the disclosed method 200 of vaporizing apparel may be that it can be used for step 210 of creating customized natural aromas or nutrients which create the process of therapeutic vaporized apparel. As such, with the disclosed method 200 of vaporizing apparel, a homeopathic method may be provided for the creation of aromas and nutrients made applicable to various articles of apparel with the intent to influence a therapeutic effect upon the occupant.

Another feature of the disclosed method 200 of vaporizing apparel may be that it can be used for step 212 of allowing the occupant to have the ability to interact with their sense of smell and taste, giving the occupant a tangible mechanism to stimulate behavior and taste, wherein the occupant has the ability to internalize nutrients through absorption and inhalation, and wherein, the occupant has the ability to externalize nutrients which is utilized for destabilizing airborne pathogens, smoke and bacteria hands-free.

In the specification and/or figures, typical embodiments of the disclosure have been disclosed. The present disclosure is not limited to such exemplary embodiments. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

The foregoing description and drawings comprise illustrative embodiments. Having thus described exemplary embodiments, it should be noted by those skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method. Many modifications and other embodiments will come to mind to one skilled in the art to which this disclosure pertains having the benefit of the teachings presented in the foregoing descriptions and the associated drawings. Although specific terms may be employed herein, they are used in a generic and descriptive sense only and not for purposes of limitation. Accordingly, the present disclosure is not limited to the specific embodiments illustrated herein but is limited only by the following claims.

1. An apparel vaporizing device for creating vaporized apparel comprising:
an enclosure with an inside configured to hold at least one apparel item;
a hanging mechanism positioned inside of the enclosure, the hanging mechanism is configured for hanging at least one of the at least one apparel item thereon in the inside of the enclosure;
a vapor container in communication with the inside of the enclosure, the vapor container is configured for providing a vapor; and
a pump configured to move the vapor from the vapor container throughout the inside of the enclosure and onto each of the at least one apparel item inside of the enclosure.

2. The apparel vaporizing device of claim 1 being configured to vaporize each of the at least one apparel item inside of the enclosure via the vapor from the vapor container, wherein the vapor from the vapor container is configured to vaporize each of the at least one apparel item with customized natural aromas, nutrients, or a combination thereof, wherein the vapor from the vapor container is configured to:
create therapeutic vaporized apparel from each of the at least one apparel item inside the enclosure;
provide a deodorizer for each of the at least one apparel item inside of the enclosure;
provide an alternative dry-cleaning of each of the at least one apparel item inside of the enclosure;
provide organic infusion to each of the at least one apparel item inside of the enclosure; and
a combination thereof.

3. The apparel vaporizing device of claim 1, wherein the enclosure is a wooden cabinet structure, wherein the wooden cabinet structure including:
a width;
a height;
a depth; and
wherein, the width, the height, and the depth are sized to accommodate each of the at least one apparel item hung on the hanging mechanism in the enclosure.

4. The apparel vaporizing device of claim 3, wherein the wooden cabinet structure including:
the width of approximately 4 feet;
the height of approximately 6 feet; or
the depth of approximately 2.5 feet.

5. The apparel vaporizing device of claim 1, wherein the enclosure including at least one door, the at least one door is configured to open for accessing the inside of the enclosure for inserting or removing each of the at least one apparel item, and the at least one door is configured to close for sealing the inside of the enclosure for vaporizing each of the at least one apparel item on the inside of the enclosure.

6. The apparel vaporizing device of claim 5, wherein:
the at least one door including 2 outswing style doors on a front side of the enclosure;
each of the at least one doors is made of a transparent material configured for viewing each of the at least one apparel item positioned on the inside of the enclosure;
the transparent material of the at least one door is glass; and
a combination thereof.

7. The apparel vaporizing device of claim 5, wherein the at least one door extends from approximate a top of the enclosure to a mid-portion of the enclosure, wherein a bottom portion of the inside of the enclosure does not include a door or opening, wherein the bottom portion may be configured for holding non-hanging apparel items that rest on a bottom of the enclosure; and
wherein the front side of the enclosure including a front member extending from the bottom of the enclosure up to the mid-portion of the enclosure.

8. The apparel vaporizing device of claim 1, wherein the hanging mechanism is a rod, wherein the rod is positioned approximate a top of the enclosure and extending from a left side of the enclosure to a right side of the enclosure.

9. The apparel vaporizing device of claim 1, wherein the vapor container is positioned on an outside of the enclosure and communicates with the inside of the enclosure through a vapor port.

10. The apparel vaporizing device of claim 9, wherein the pump is positioned in the vapor port for moving the vapor from the vapor container through the vapor port and into the inside of the enclosure.

11. The apparel vaporizing device of claim 10, wherein the pump is a propeller fan positioned in the vapor port.

12. The apparel vaporizing device of claim 9, wherein the vapor container is positioned on an exterior shelf mounted on the outside of the enclosure; and
wherein the exterior shelf including a housing configured to seal the vapor container inside of the housing on the exterior shelf mounted on the outside of the enclosure.

13. The apparel vaporizing device of claim 12, wherein:
the housing of the exterior shelf including an access panel on one side of the housing, the access panel is configured to open for accessing the inside of the housing for inserting, removing, or re-filing the vapor container, and the access panel is configured to close for sealing the inside of the housing with the vapor container therein;
the access panel is made from a transparent material;
the transparent material of the access panel is glass;
the exterior shelf including an angled support bracket configured to support the exterior shelf on the outside of the enclosure; and
a combination thereof.

14. The apparel vaporizing device of claim 1, wherein the vapor container is an aroma lamp, an evaporative diffuser, a heat diffuser, a nebulizer, or an ultrasonic diffuser.

15. The apparel vaporizing device of claim 1, wherein the vapor container is an ultrasonic diffuser.

16. The apparel vaporizing device of claim 1 further comprising a low voltage battery, the low voltage battery is configured to power the vapor container and the pump for moving the vapor from the vapor container throughout the inside of the enclosure and onto each of the at least one apparel item inside of the enclosure.

17. An apparel vaporizing device for creating vaporized apparel comprising:
an enclosure with an inside configured to hold at least one apparel item, the enclosure is a wooden cabinet structure including a width, a height, and a depth, wherein, the width, the height, and the depth are sized to accommodate each of the at least one apparel item in the inside of the enclosure;
the enclosure including at least one door, the at least one door is configured to open for accessing the inside of the enclosure for inserting or removing each of the at least one apparel item, and the at least one door is configured to close for sealing the inside of the enclosure for vaporizing each of the at least one apparel items on the inside of the enclosure, the at least one door including 2 outswing style doors on a front side of the enclosure, each of the at least one doors is made of a transparent material configured for viewing each of the at least one apparel item positioned on the inside of the enclosure, the at least one door extends from approximate a top of the enclosure to a mid-portion of the enclosure, a bottom portion of the enclosure does not include a door or opening, the bottom portion may be configured for holding non-hanging apparel items that rest on a bottom of the enclosure, the front side of the enclosure including a front member extending from the bottom of the enclosure up to the mid-portion of the enclosure
a hanging mechanism positioned inside of the enclosure, the hanging mechanism is configured for hanging at least one of the at least one apparel item thereon in the inside of the enclosure, the hanging mechanism is a rod positioned approximate a top of the enclosure and extending from a left side of the enclosure to a right side of the enclosure;
a vapor container in communication with the inside of the enclosure, the vapor container is configured for providing a vapor, the vapor container is an ultrasonic diffuser;
a pump configured to move the vapor from the vapor container throughout the inside of the enclosure and onto each of the at least one apparel item inside of the enclosure;
the vapor container is positioned on an outside of the enclosure and communicates with the inside of the enclosure through a vapor port, wherein the pump is positioned in the vapor port for moving the vapor from the vapor container through the vapor port and into the inside of the enclosure, wherein the pump is a propeller fan positioned in the vapor port;
the vapor container is positioned on an exterior shelf mounted on the outside of the enclosure; the exterior shelf including a housing configured to seal the vapor container inside of the housing on the exterior shelf mounted on the outside of the enclosure, the housing of the exterior shelf including an access panel on one side of the housing, the access panel is configured to open for accessing the inside of the housing for inserting, removing, or re-filing the vapor container, and the access panel is configured to close for sealing the inside of the housing with the vapor container therein, wherein the access panel is made from the transparent material, wherein the exterior shelf including an angled support bracket configured to support the exterior shelf on the outside of the enclosure;
a low voltage battery, the low voltage battery is configured to power the vapor container and the pump for moving the vapor from the vapor container throughout the inside of the enclosure and onto each of the at least one apparel item inside of the enclosure;
wherein, the apparel vaporizing device is configured to vaporize each of the at least one apparel item inside of the enclosure via the vapor from the vapor container;
wherein the vapor from the vapor container is configured to:
vaporize each of the at least one apparel item with customized natural aromas, nutrients, or a combination thereof;
create therapeutic vaporized apparel from each of the at least one apparel item inside the enclosure;
provide a deodorizer for each of the at least one apparel item inside of the enclosure;
provide an alternative dry-cleaning of each of the at least one apparel item inside of the enclosure;
provide organic infusion to each of the at least one apparel item inside of the enclosure; and
a combination thereof.

18. A method of vaporizing apparel comprising:
providing an apparel vaporizing device comprising:
an enclosure with an inside configured to hold at least one apparel item;
a hanging mechanism positioned inside of the enclosure, the hanging mechanism is configured for hanging at least one of the at least one apparel item thereon in the inside of the enclosure;
a vapor container in communication with the inside of the enclosure, the vapor container is configured for providing a vapor;
a pump configured to move the vapor from the vapor container throughout the inside of the enclosure and onto each of the at least one apparel item inside of the enclosure;
inserting the at least one apparel item into the inside of the enclosure; and
vaporizing the at least one apparel item by moving vapor from the vapor container throughout the inside of the enclosure.

19. The method of claim 18, wherein the inserting of the at least one apparel item into the inside of the enclosure including hanging at least one of the at least one apparel item on the hanging mechanism inside of the enclosure.

20. The method of claim 18 further including:
creating customized natural aromas or nutrients which create a process of therapeutic vaporized apparel, wherein a homeopathic method is provided for creation of aromas and nutrients made applicable to various articles of apparel with an intent to influence a therapeutic effect upon an occupant; or
allowing the occupant to have an ability to interact with their sense of smell and taste, giving the occupant a tangible mechanism to stimulate behavior and taste, wherein the occupant has the ability to internalize nutrients through absorption and inhalation, and wherein, the occupant has the ability to externalize nutrients which is utilized for destabilizing airborne pathogens, smoke and bacteria hands-free.
